(19) United States
(12) Patent Application Publication
(10) Pub. No.: US 2008/0208238 A1
(43) Pub. Date: Aug. 28, 2008

(54) Reducing Needle/Multi-Sided Needle

(76) Inventor: Scott Brown, Haymarket, VA (US)

Correspondence Address: WILSON DANIEL SWAYZE, JR., 3804 CLEARWATER CT., PLANO, TX, 75025, US

(21) Appl. No.: 12/037,461

(22) Filed: Feb. 26, 2008

Related U.S. Application Data
(60) Provisional application No. 60/891,870, filed on Feb. 27, 2007.

(51) Int. Cl. A61B 17/34

(52) U.S. Cl. 606/189

(57) ABSTRACT

A reducing needle for use by an acupuncturist may include a handle member for the reducing needle to be handled, and the reducing needle may include a central bore to allow material to be withdrawn from the patient. The reducing needle may include a lower aperture to connect to the central bore, and the reducing needle may include an upper aperture to connect with the central bore. An acupuncture needle for use by an acupuncturist may include a handle to allow the acupuncturist to hold the acupuncture needle, and a needle portion connected to the handle may have a least two linear surfaces. The needle portion may include at least three linear surfaces, and the needle portion may include at least four linear surfaces. The needle portion may include at least six linear surfaces.

### PRIORITY

The present invention claims priority under 35 USC section 119 based upon provisional application 60/891,870 which was filed on Feb. 27, 2007.

### BACKGROUND OF THE INVENTION

The invention relates to a device for stimulating acupuncture points of a body and, more particularly, for stimulating them from the surface of the body.

Acupuncture is an ancient medical art dating back more than 2000 years.

The present invention relates generally to the stimulation of acupuncture points to effect medical treatment. The present invention relates particularly to the stimulation of acupuncture points to treat a variety of medical disorders.

For more than three thousand years, Chinese physicians have used acupuncture to alleviate pain and treat a myriad of diseases. While the principles underlying the effectiveness of acupuncture as a treatment form are not yet completely understood, it has been well recognized that the stimulation of certain points on the human body have therapeutic effects. Such stimulation may be effected through the insertion of needles, activation of surface electrodes, or other means.

Traditionally, points in the human body (herein called acupuncture points) were reached by piercing the body with fine wires or needles. The needles were then rotated or oscillated about their longitudinal axes, for example by rubbing an exposed end of a needle between a thumb and finger, to stimulate the acupuncture points.

Stimulation of specific acupuncture points has been shown in several studies to have great utility as adjunct therapy to conventional medical treatments such as nausea and vomiting, postoperative pain, headache, smoking cessation, erectile dysfunction, depression, male infertility, dysmenorrhea, and stomach acid secretion. Great impetus to the use of acupuncture was generated when the National Institutes of Health Consensus Conference on Acupuncture in 1997 showed efficacy of acupuncture in adult postoperative and chemotherapy nausea and vomiting and in postoperative dental pain, and that there were other situations, such as addiction, stroke rehabilitation, headache, menstrual cramps, tennis elbow, fibromyalgia, myofascial pain, osteoarthritis, low back pain, carpal tunnel syndrome, and asthma, in which acupuncture may be useful as an adjunct treatment or an acceptable alternative or be included in a comprehensive management program.

The practice of acupuncture is based on the theory that certain lines running across the human body, referred to as meridians, are each associated with a certain organ. For example, the Lung Meridian (designated “LU”) runs from the tip of the thumb, along the forearm above the radius, and up to the shoulder. Other meridians include the Liver Meridian (“LR”), Stomach Meridian (“ST”), Spleen Meridian (“SP”), Heart Meridian (“HT”), and Pericardium Meridian (“PC”). Other abbreviations include Liver (“LV”), Gallbladder (“GB”), Kidney (“KD”), Urinary Bladder (“UB”), Spleen (“SP”), San Jiao (“SJ”), Large Intestine (“LI”), Small Intestine (“SI”) Along these meridians are certain points that, when stimulated, have certain therapeutic effects on the human body. Points along a meridian are designated by the meridian symbol combined with a number for that point; for example, the point on the Lung Meridian that lies just above the joint between the radius and the humerus is designated “L5”. The stimulation of each point along a meridian is believed to have an effect different from the stimulation of other points along the same meridian. Thus according to acupuncture theory, the correct location of the proper meridian, and the correct location of the proper point along that meridian, is crucial to effecting a proper treatment. In particular, the traditional acupuncture treatment to relieve anxiety-related disorders requires the insertion of needles at least two specific points. A standard reference work identifying the acupuncture meridians and points, and the believed effects of stimulating many of those points, is Charles A. Meeker, Acupuncture for the Practitioner or Advanced Student (3d Ed. 1979), which is incorporated by reference herein.

The use of acupuncture or the stimulation of acupuncture points for the general purposes of medical treatment and research has continued to the present day. The following prior art references demonstrate modern developments in this area:

U.S. Pat. No. 3,908,669 issued to Man et al. is of interest for the prior art of devices using acupuncture in medical research and therapy. Man et al. discloses the use of electrically-charged needles rather than electrodes.

U.S. Pat. No. 4,981,146 issued to Bertolucci discloses a nausea control device for mounting onto the human wrist. Electrodes stimulate the pericardium six (or PC6) acupuncture point.

U.S. Pat. No. 5,269,304 issued to Matthews discloses an electro-therapy apparatus including at least two electrodes. Matthews suggests the efficacy of higher frequencies up to around 200 kHz, but also suggests employing slightly different frequencies on the two electrodes so as to achieve beat frequencies of 80 to 130 Hz.

U.S. Pat. No. 4,055,190 issued to Tany discloses an electrical therapeutic apparatus for applying selected voltages and frequencies to various acupuncture meridians through needles. Suggested frequencies for each meridian are disclosed ranging from 500 Hz to 200 kHz.

U.S. Pat. No. 5,417,706 issued to Chun discloses a method of treating various types of baldness by inserting acupuncture needles into selected acupuncture points on multiple meridians. Chun identifies twelve particular acupuncture points on eight different meridians as effective in the therapy.

It is known, therefore, in the prior art to employ surface stimulation over acupuncture points to alleviate various medical conditions. It is also known to employ multiple acupuncture points on multiple meridians to treat various conditions of baldness. It is also known that the stimulation of specific acupuncture points with needles may be used to treat anxiety and related disorders. In particular, the traditional prior art acupuncture treatment for anxiety and related disorders was the insertion of needles at least two specific points.

U.S. Pat. No. 6,961,622 discloses a device, which is preferably disc shaped, has two primary layers. A first layer has one side, which is the lower side in use, for adhering to a patient's skin surface, a second, upper layer on the underside of which electrical circuitry is printed or affixed so that the electrical circuitry is sandwiched between the layers. The device can be made in several sizes to accommodate patient size and location of the acupuncture point to be stimulated. Two distinct forms of the device are presented: a single use and a reusable device for. In the single use device, the first, lower layer is preferably a foam with non-conductive adhesive on both sides: the bottom side for adhering to the skin and the top side for adhering to the upper polyester disc. The holes through this lower foam layer include, preferably, eight holes spaced concentrically about one central hole in the middle of the lower foam layer. All these holes are, preferably, overfilled with an electrically conductive gel that projects from the lower foam layer. The conductive circuitry preferably printed on the underside of the upper layer provides a series connection to the gel in each of the eight concentric holes and a separate connection to the gel in the center hole when the upper layer is adhered to the lower layer. The printed circuitry may be a silver/silver chloride polymer film, also provides for a tab(s) which can be permanently or temporarily affixed to an integral or remote simulator through direct contact or wire leads. In the reusable device a pressure-sensitive adhesive material forms the lower layer which allows for multiple applications to a patient's skin. The adhesive lower layer is transparent to show the circular central electrode the annular electrode. The electrodes are preferably silver/silver chloride polymer film. In either configuration, metal core insulated leads can be use for electrical connection with the opposite ends of the leads connected to jacks for connection to an impulse stimulator or can end in electrically conductive tabs.

U.S. Pat. No. 5,950,635 discloses a method of treating various anxiety-related disorders, such as Post-Traumatic Stress Disorder, panic attacks, or general anxiety disorder is disclosed. It has been found that the P1 midlatency auditory evoked potential of such patients undergoes increased amplitude and decreased habituation. Stimulation of three specific acupuncture points LR3, HT3, and PC6) on three different acupuncture meridians has been found to reduce the amplitude of the P1 potential using 20-minute periods of stimulation. Low frequency stimulation (around 5 Hz) was found to be most effective. Repeated periods of stimulation were also found to increase the effect of the treatment.

### SUMMARY

A reducing needle for use by an acupuncturist may include a handle member for the reducing needle to be handled, and the reducing needle may include a central bore.

The reducing needle may include a lower aperture to connect to the central bore, and the reducing needle may include an upper aperture to connect with the central bore.

An acupuncture needle for use by an acupuncturist may include a handle to allow the acupuncturist to hold the acupuncture needle, and a needle portion connected to the handle may have a least two linear surfaces.

The needle portion may include at least three linear surfaces, and the needle portion may include at least four linear surfaces.

The needle portion may include at least six linear surfaces.

### BRIEF DESCRIPTION OF THE DRAWINGS

The invention may be understood by reference to the following description taken in conjunction with the accompanying drawings, in which, like reference numerals identify like elements, and in which:

FIG. 1 illustrates a side view of a reducing needle of the present invention;

FIG. 2 illustrates a side view of the reducing needle of the present invention under the skin of a patient;

FIG. 3 illustrates a side view of an acupuncture needle having multi-sides.

FIG. 4 illustrates a bottom view of a six sided acupuncture needle;

FIG. 5 illustrates a bottom view of the three sided acupuncture needle;

FIG. 6 illustrates a bottom view of a four sided acupuncture needle.

### DETAILED DESCRIPTION

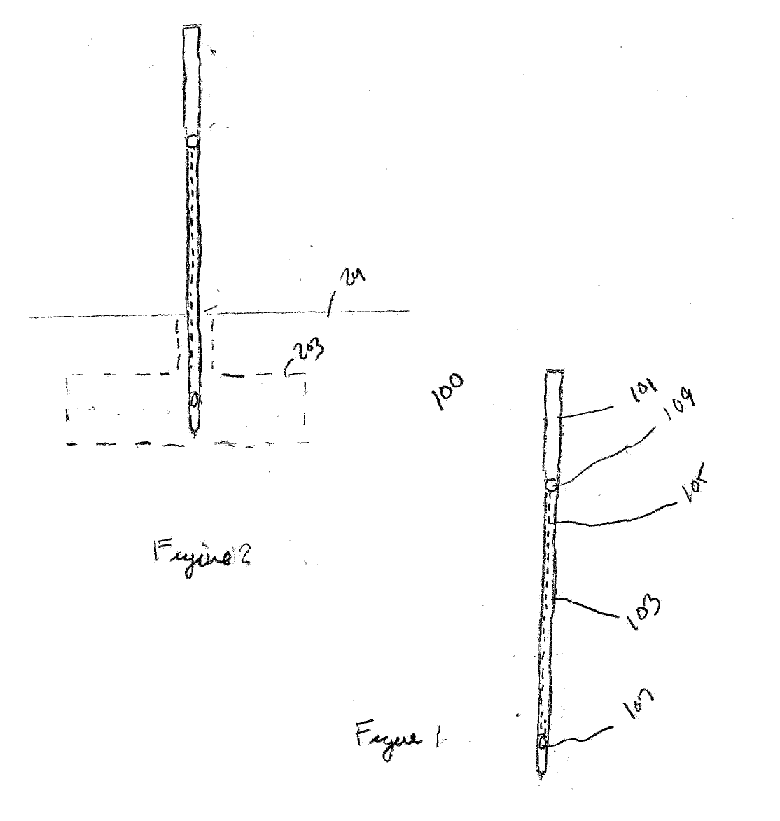

FIGS. 1 and 2 illustrate a reducing needle of the present invention. The reducing needle of the present invention can be formed in varying lengths and gauges and may be made from materials that are currently used such as steel and other types of rigid materials. The reducing needle 100 of the present invention may be used in the same way that a normal acupuncture needle is used.

FIG. 1 illustrates the reducing needle 100 of the present invention. The reducing needle 100 includes a handle member 101 to allow the user of the reducing needle 100 to manipulate the reducing needle 100. A lower aperture 107 is positioned near the distal end of the reducing needle 100 and cooperates with a substantially central bore member 103 which extends in the longitudinal direction of the reducing needle 100 to form a passageway for delivery and removal of material such as fluids (or gases/solids) form the patient. The central bore member 103 extends to the upper aperture 109 to complete the passageway between the lower aperture 107 and the upper aperture 109. The upper aperture 109 is located near the proximal end of the reducing needle 100. The reducing needle 100 may come in different lengths and gauges.

FIG. 2 illustrates that the reducing needle 100 has penetrated the skin layer 201 and the lower aperture 107 has entered a region 203 which includes excess pathogenic Qi or other material which should be treated or removed. The upper aperture 109 remains above the skin layer 201 to provide access to the lower aperture 107 by virtue of the central bore member 103.

FIG. 3 illustrates an acupuncture needle 300 which is multi-sided. FIG. 3 illustrates a side view of the acupuncture 300 which allows the acupuncturist to tonify an acupuncture point which coincides with the traditional Chinese medical theory. The acupuncture needle 300 can be used to provide different therapeutic results based upon the point prescription of the acupuncturist.

FIG. 3 illustrates a handle 309 which is connected to a needle portion 311 which includes a first needle surface 307 adjacent to a second needle surface 308 and defined by a needle edge 305. The needle portion 311 has at least two needle surfaces being substantially linear. The needle surface 307, 308 which are substantially linear and the needle edge 305 are substantially longitudinal to the acupuncture needle 300 and form a point at the distal end of the acupuncture needle 300.

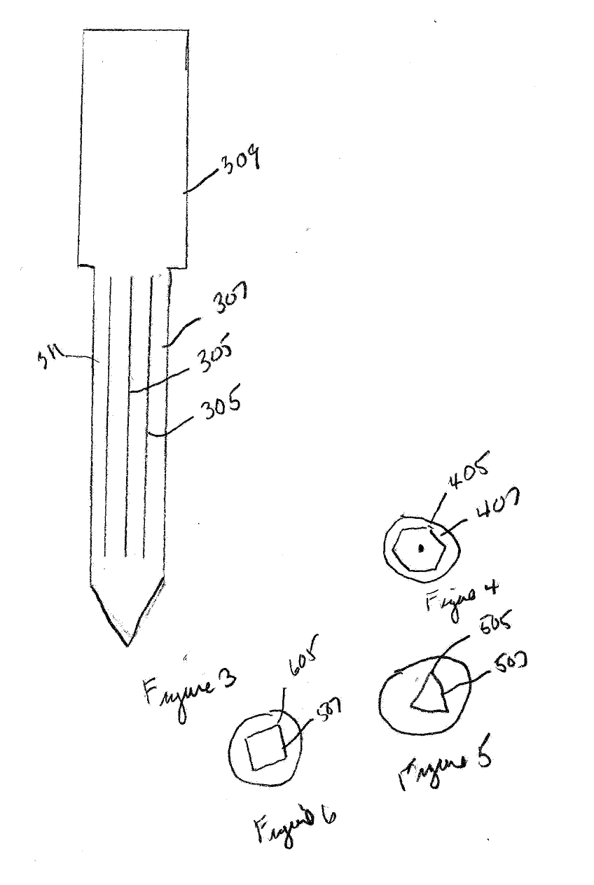

FIG. 4 illustrates a six sided needle which includes needle side 407 and needle edge 405.

FIG. 5 illustrates a three sided needle which includes needle side 507 and a needle edge 505.

FIG. 6 illustrates a four sided needle which includes needle side 607 and needle edge 505.

The length and gauge of the needle can be varied accordance with the needs of the acupuncturist as well as the number of sides.

While the invention is susceptible to various modifications and alternative forms, specific embodiments thereof have been shown by way of example in the drawings and are herein described in detail. It should be understood, however, that the description herein of specific embodiments is not intended to limit the invention to the particular forms disclosed.

1. A reducing needle for use by an acupuncturist, comprising:
a handle member for the reducing needle to be handled;
wherein the reducing needle includes a central bore.

2. As in claim 1, wherein the reducing needle includes a lower aperture to connect to the central bore.

3. As in claim 2, wherein the reducing needle includes an upper aperture to connect with the central bore.

4. An acupuncture needle for use by an acupuncturist, comprising:
a handle to allow the acupuncturist to hold the acupuncture needle;
a needle portion connected to the handle having a least two linear surfaces.

5. as in claim 4, wherein the needle portion includes at least three linear surfaces.

6. As in claim 5, wherein the needle portion includes at least four linear surfaces.

7. As in claim 6, wherein the needle portion includes at least six linear surfaces.
